(19) United States
(12) Patent Application Publication
(10) Pub. No.: US 2005/0131726 A1
(43) Pub. Date: Jun. 16, 2005

(54) Turn-key mediation system and method

(76) Inventor: Angelique Bartholomew, Missouri City, TX (US)

Correspondence Address: Delphine M. James;Suite 170, 2656 South Loop West, Houston, TX, 77054, US

(21) Appl. No.: 10/974,352

(22) Filed: Oct. 27, 2004

Related U.S. Application Data
(60) Provisional application No. 60/514,756, filed on Oct. 27, 2003.

(51) Int. Cl. G06F 17/60; A61K 31/275; A01N 37/34; G09B 25/00

(52) U.S. Cl. 705/1; 434/365

(57) ABSTRACT

The present invention discloses a system and method of resolving conflict between two parties who are members of a designated school system, or who are members of a religious organization, or who are members of a community organization. The system include generating and maintaining a mediator database containing a plurality of mediators in which each mediator is designated to support a specific market area, receiving from a specific market area a request to resolve a conflict between the two parties, identifying from the coordinator database a market coordinator that support the market area, transmitting to the market coordinator a request for a mediator, selecting by the market coordinator from the mediator database a mediator for the specific market area, generating a schedule for mediation for the specific market area, transmitting to the market area and each party the mediation schedule, collecting from the mediator each party position on the conflict; and generating a conflict resolution agreement based upon the position of each party.

Applicant claims the benefit of provisional patent Ser. No. ______

### BACKGROUND

The mediation process is utilized in the court system today. However, formal mediation techniques cannot be utilized in school and community to resolve conflicts between students, teachers and parents. This system and method provides a tailored system and method to support conflict resolution in schools, communities, churches, and youth organizations. Currently, utilized in schools is a peer-to-peer mediation system which may not be as effective because the students in conflict may not feel comfortable being completely open with a fellow student.

### SUMMARY

The system provides the capability of managing the mediator selection process through the school and a method of managing the conflict resolution or mediation process between the two students with conflict. The school requests mediation, the mediation request is confirmed, and a mediator is selected. The mediation process includes specific techniques for forcing communication between the parties and creating a workable resolution between both parities. After the mediation process is complete, the system can include a skill building component for each student.

### BRIEF DESCRIPTION OF DRAWINGS

FIG. 1 is a block diagram of the overall mediation system.

FIG. 2 is a detail block diagram of the specific mediation process.

FIG. 3 is a sample mediation request form.

FIG. 4 is a sample mediation confirmation form.

FIG. 5 is a sample notice to parent form.

FIG. 6 is a sample resolution agreement.

FIG. 7 is one embodiment of the mediation system implemented as an automated system.

FIG. 8 is one embodiment of the system showing the objects to implement the system.

FIG. 9 is one embodiment of the system showing the objects to implement the skill building component.

### DETAILED SPECIFICATION

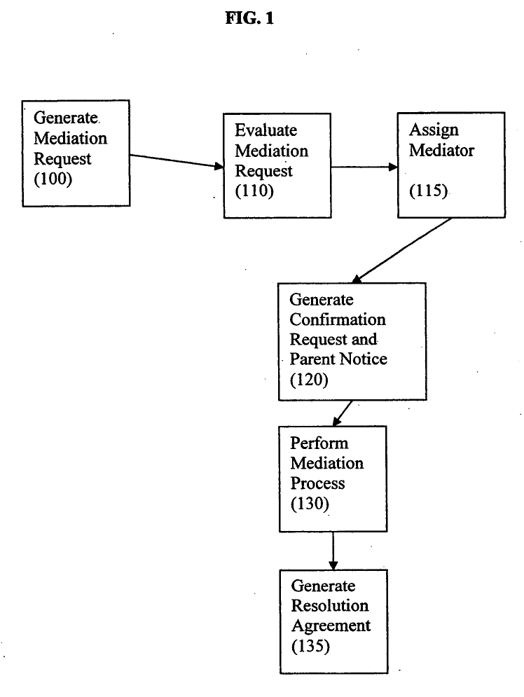

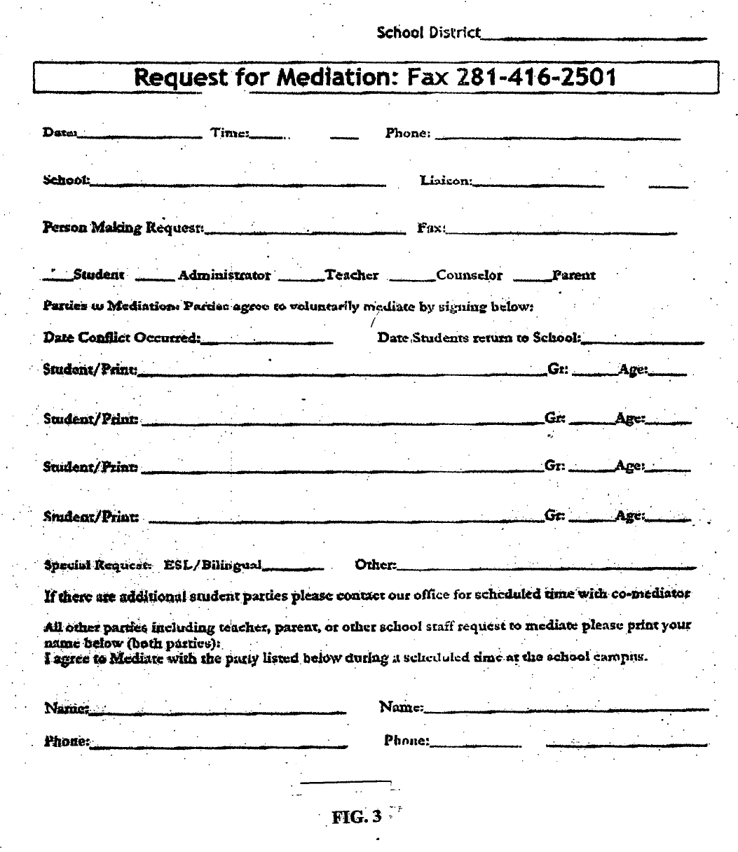

Referring to FIG. 1, there is shown one embodiment of the overall business method of the present invention. At step 100, the school generates a request for mediation. The request comprises pertinent school information as well as child information. A sample mediation request is illustrated in FIG. 3. At step 100, the school sends the request to a Market Coordinator. The Market Coordinator is responsible for a specific area Each area is assigned a specific group of mediators which report to the Market Coordinator. A list of mediators is maintained by the market coordinator based upon the type of conflict and the availability of the mediator.

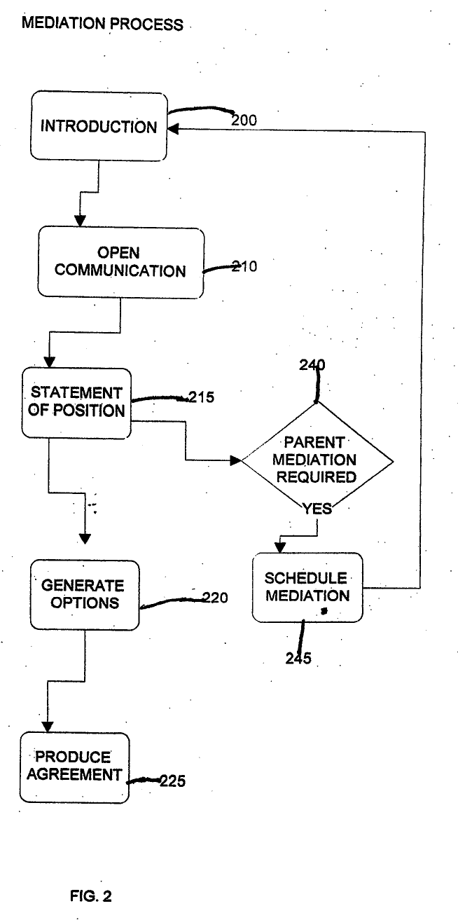

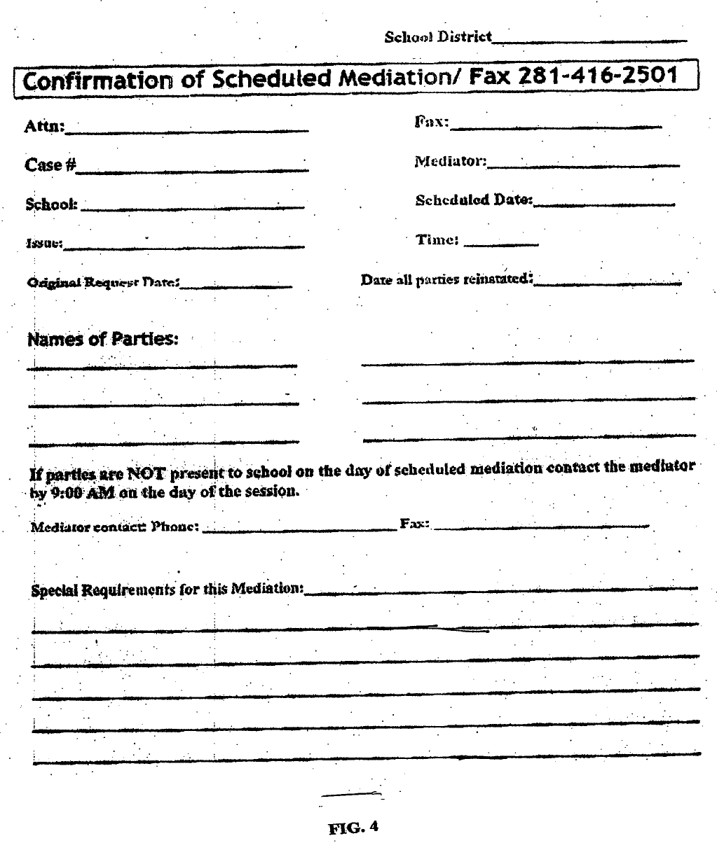

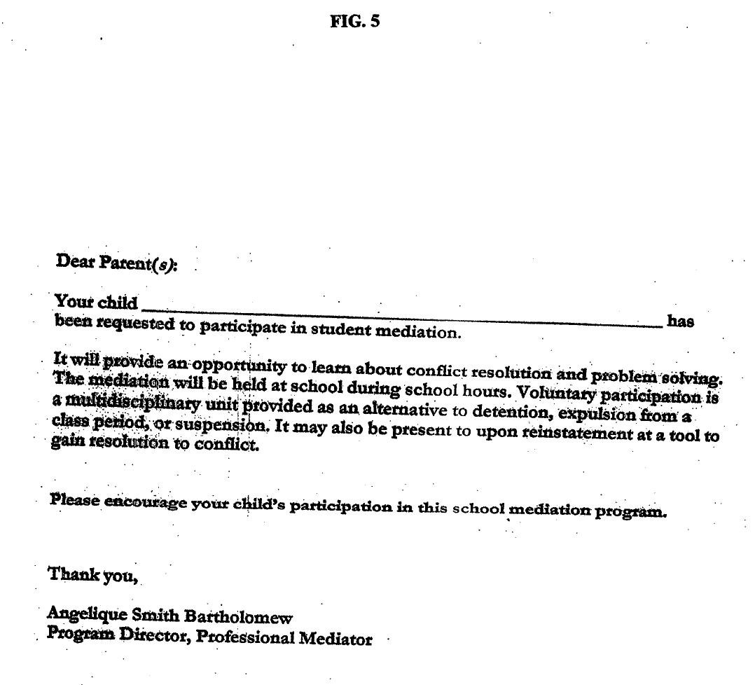

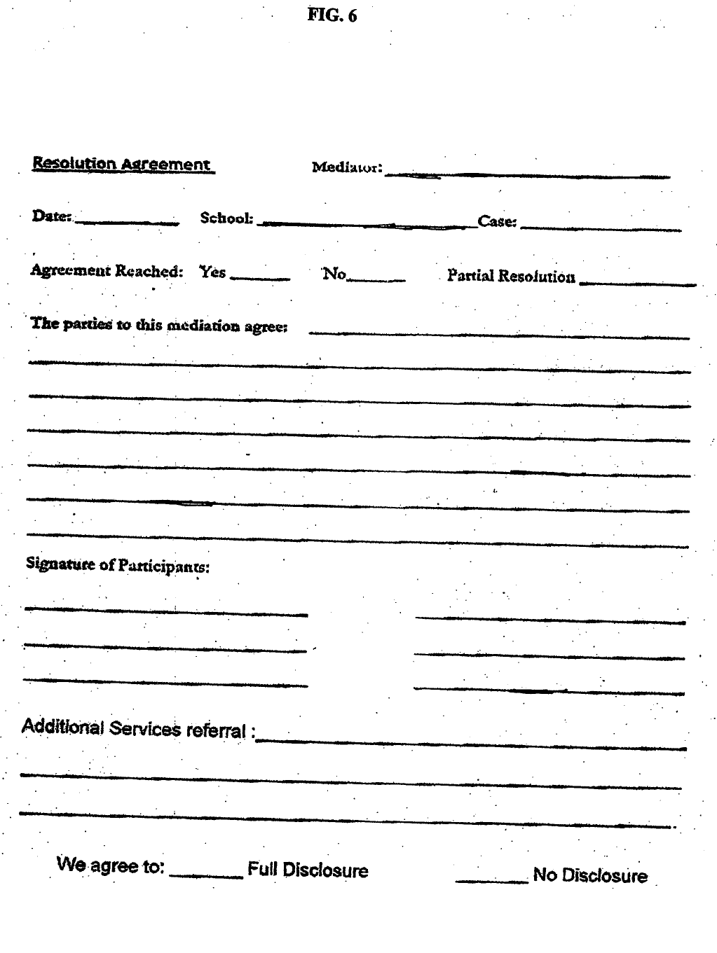

At step 110 the market coordinator evaluates the mediation request. If additional information is required by the market coordinator to process the mediation request, the market coordinator contacts the school about the conflict issues between the parties. At step 115, based upon the conflict issues, the Market Coordinator will appoint an appropriate mediator based upon their experience and availability. At step 120, a confirmation notice and a parent notice are generated by the Market Coordinator and sent to the school. A sample parent notice is depicted in FIG. 5. The confirmation notice is sent to the school confirming the appointment of a mediator and providing a scheduled time for mediation. A sample confirmation notice is depicted in FIG. 4. At step 130, the parties mediate as depicted in FIG. 2. In step 130, the mediator establishes each party position. Then, the mediator makes inquires to each party to resolve the conflict. At step 135, upon completion of mediation, the mediator generates the agreement between the two parties. A sample agreement is depicted in FIG. 6.

Referring to FIG. 2, there is shown a detail functional block diagram of the mediation process. At step 200, the mediator begins the process by outlining the rules of engagement between the two parties in conflict. At step 210, the mediator starts the process of opening communication between the two parties. In this step, in a face-to-face stance, each party states their position. Then, each party must analyze the other party position on the conflict. After each party performs the analysis, so the two the parties can begin to resolve the conflict.

At step 215 in a back-to-back stance, each party begins to intensely reveal their position. In this step the mediator ask a series of questions related to the conflicts. With these specific questions, each party must reveal how they feel about the situation and why they feel the way that they do. Additionally, each party must state whether they feel that they belong to their school community. Next, each party is asked a series of questions to help them understand the conflict versus violence styles. Lastly, each party is asking a series of question to help them exchange roles. The purpose of this step is for each party to better understand each other's position and enter a stage of feeling remorseful.

Additionally, at step 215, an analysis is done to determine whether the parents of the parties need to be brought in for independent mediation. If yes, then a request is sent to the market coordinator to schedule parent mediation. At step 245, the steps in FIG. 1 are repeated for the parent. Then, the parent enters the mediation process at step 200.

At step 220, each party generates at least one resolution option. Each generated option is analyzed by both parties to determine whether it is a workable resolution. After each option is analyzed, an agreement is generated by the mediator based upon only the workable resolutions. FIG. 6 is a sample of a Resolution Agreement.

As part of the resolution agreement, skill building sessions can be established for each student, parent, or teacher. Thus, after completion of the conflict resolution or mediation process in step 220, as part of the resolution agreement the system provides conflict resolution educational instruction and skill building to the parties that went through the mediation process. The skill building component utilizes progressive scenarios levels in at least two different levels of conflict resolutions. Each level consists of several domain areas. Within each domain area special conflict resolution scenarios are performed by the student. The system tracks the progression through each domain area. After the student completes each level, then a special program is designed for the student to ensure specialized skill building. The levels and domains are as follows:
1. Conflict Resolution Education and Practices
Domain Areas (Vocabulary, Mediator, Bystander, Participant)
2. Interpersonal Conflict
Domain Areas (Conflict Personalities, Conflict with Self, Conflict with others i.e. adults, friend, and family)
3. Community Conflict
Domain Areas (School, Neighborhood, World-Wide)
4. Empowering Resolution.
Domain Areas (Self-Expression, Decision Making, Critical Thinking)

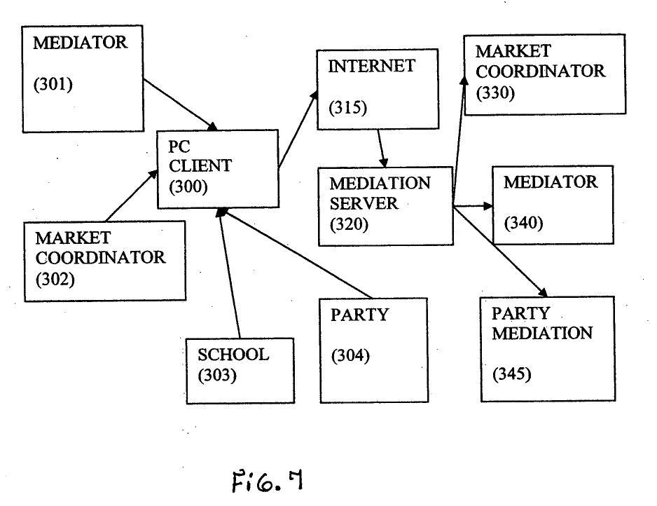

Referring to FIG. 7, there is shown a block diagram of one embodiment of the present invention, implemented in a client/server model over a network. Network (315) can be LAN, WAN, the Internet or an Intranet. Client software (300) can be an Internet Service Provider browser or proprietary software designed to access the server software. A PC, laptop, notebook, workstation or another suitable system can be utilized to execute the client software (300). At least one mediation server (320) implements the functions illustrated in FIG. 1. Several clients can access the system. The system can provide a user interface for the following clients:
a party client (304) to do skill building
a market coordinator client (302) to schedule and confirm mediation
a mediator client (301) to setup the list and generate the resolution
a school client (303) to generate the mediation request

Several databases can be developed to support this system. Database (330) of market information stores school to market coordinator information. Database (340) of mediator information stores mediator to market coordinator information. Database (345) of party mediation stores information about the parties involved in the conflict and the generated agreement.

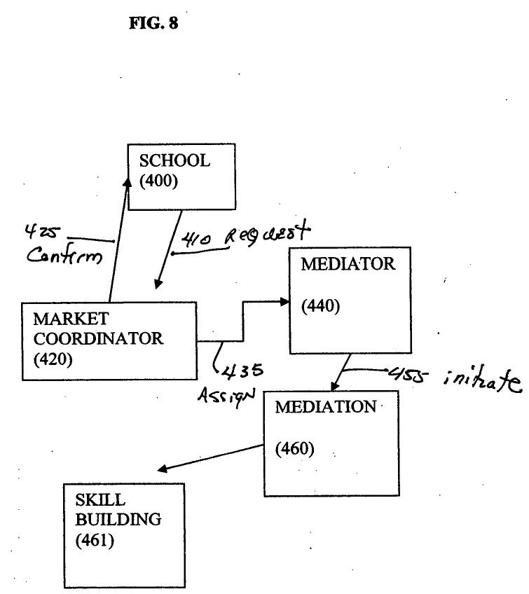

Referring to FIG. 8, there is shown one embodiment of the object modules required to implement the Conflict resolution process (320):
School object (400) contains the data and operations to support mediation request (410) and acknowledgement of mediation confirmation (425).
Market Coordinator object (420) contains data and operations to support accepting mediation request (410), issuing confirmation (425) and issuing notice (430) to parent object (450), and assigning mediator (435).
Mediator object (440) contains data and operations to support accepting assignment (440) and initiating mediation (455)
Mediation object (460) contains data and operations to support the generation of the resolution agreement based upon the data gathered in the mediation process by the mediator.
Skill building object (461) contains the data and operations to support the party mediation.

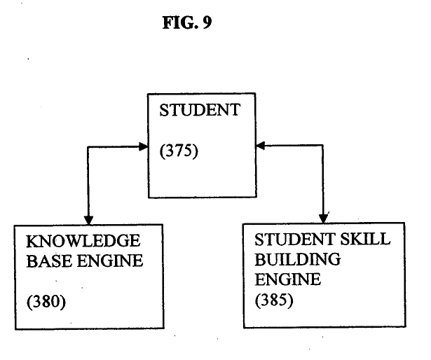

Referring to FIG. 9, there is shown one embodiment of the object modules required to implement the skill building process (461).
Student Module (375) which contains the data and operations to authenticate the student to the system, to track the progression level of the student in the domain area of a particular skill building level within the knowledge base engine as well as within the student skill building engine, and to track which scenarios the student has failed in each domain area
Knowledge base engine (380) which contains the base scenarios for each domain area of each skill building level.
Student skill building engine (385) which contains the scenarios that the student has failed within each domain area of each skill building level.

The present invention is not limited to the software design structure as described above.

1. A method of resolving conflict between two parties who are members of a designated school system, or who are members of a religious organization, or who are members of a community organization, the method comprising the steps of:
identifying a market coordinator for a specific market area;
generating and maintaining a list of mediators to support a specific market area;
receiving from a specific market area a request to resolve a conflict between the two parties;
transmitting to the market coordinator a request for a mediator;
selecting from the list of mediators a mediator to perform mediation;
generating a confirmation notice for mediation;
transmitting to the market area the confirmation notice;
while performing mediation, determining by the mediator each parties' position on the conflict; and
generating a conflict resolution agreement based upon the position of each party.

2. The method of claim 1 wherein the market area is a defined school.

3. The method of claim 1 wherein the market area is a community organization.

4. The method of claim 1 wherein the selecting step further comprises scheduling a mediator based upon their availability and their experience with handling a specific type of conflict resolution.

5. The method of claim 1 wherein the generation a confirmation notice step further comprises:
generating a parent notice; and
transmitting the parent notice to the school.

6. The method of claim 1 wherein the determining step further comprises:
generating a set of inquires for each party based upon their position on the conflict;
accepting responses from the set of inquires; and
generating the resolution agreement based upon the responses to the set of inquires.

7. The method of claim 1 wherein the generating of the resolution agreement further comprising:
generating a knowledge based engine based upon at least one conflict resolution level having at least one domain level; and
generating within each domain level a plurality of situational scenarios.

8. The method of claim 7 further comprising:
authenticating each party assigned to access the knowledge based engine;
displaying to each party a scenario from a selected domain area within a resolution level;
tracking each party progression through each domain area within each resolution level; and
tracking each party failure rate of each scenario in each selected domain area within a resolution level.

9. The method of claim 8 further comprising generating a party specific knowledge based engine based upon the party's failure rate of each scenario in each selected domain area within a resolution level.

10. A system of resolving conflict between two parties who are members of a designated school system, or who are members of a religious organization, or who are members of a community organization, the system comprising:
a school component operative to generate a mediation request;
a market coordinator component operative to receive the mediation request, assign a mediator to handle the mediation request, schedule the mediation between the school and the mediator and generate a notice to the school to confirm the mediation request; and
a mediator component operative to determine each parties' position on the conflict during the mediation process; and to generate a conflict resolution agreement based upon the position of each party.

11. The system of claim 10 wherein the market coordinator component further operative to schedule the mediator based upon their availability and their experience with handling a specific type of conflict resolution.

12. The system of claim 10 wherein the market coordinator component is further operative to:
generate a parent notice; and
transmitt the parent notice to the school.

13. The system of claim 10 wherein the mediator component is further operative to:
generate a set of inquires for each party based upon their position on the conflict;
accept responses from the set of inquires; and
generate the resolution agreement based upon the responses to the set of inquires.

14. The system of claim 10 further comprising a skill building component is further operative to:
generate a knowledge based engine based upon at least one conflict resolution level having at least one domain level; and
generate within each domain level a plurality of situational scenarios.

15. The system of claim 14 wherein the skill building component is further operative to:
authenticate each party assigned to access the knowledge based engine;
display to each party a scenario from a selected domain area within a resolution level;
track each party progression through each domain area within each resolution level; and
track each party failure rate of each scenario in each selected domain area within a resolution level.

16. The system of claim 15 wherein the skill building component is further operative to generate a party specific knowledge based engine based upon the party's failure rate of each scenario in each selected domain area within a resolution level.
